(19) United States
(12) Patent Application Publication
(10) Pub. No.: US 2023/0270770 A1
(43) Pub. Date: Aug. 31, 2023

(54) NUTRITIONAL SUPPLEMENTS FOR REPAIRING MUSCLE AND DEFENDING AGAINST DETERIORATION FROM HUMAN AGING

(71) Applicant: Moises HERNANDEZ-VAZQUEZ, San Diego, CA (US)

(72) Inventor: Moises HERNANDEZ-VAZQUEZ, San Diego, CA (US)

(21) Appl. No.: 18/006,980

(22) PCT Filed: Jul. 28, 2021
(86) PCT No.: PCT/US2021/043557

§ 371 Date: Jan. 26, 2023

Related U.S. Application Data
(60) Provisional application No. 63/059,000, filed on Jul. 30, 2020.

(51) Int. Cl. A61K 31/706; A61K 38/39; A61K 31/728; A61K 31/05; A61K 31/09; A61K 9/48; A61K 9/00

(52) U.S. Cl. CPC A61K 31/706; A61K 38/39; A61K 31/728; A61K 31/05; A61K 31/09; A61K 9/4825; A61K 9/0095

(57) ABSTRACT

Provided herein, inter alia, are compositions and methods for health promotion. The compositions can include at least one NAD+ intermediate, at least one stilbenoid, collagen peptides and hyaluronic acid.

### CROSS-REFERENCES TO RELATED APPLICATIONS

This application claims priority benefit to U.S. provisional 63/059,000 filed Jul. 30, 2020, which is incorporated herein in its entirety.

### TECHNICAL FIELD OF THE INVENTION

The disclosure generally relates to nutritional supplements. In particular the disclosure provides compositions and methods for reducing symptoms of aging, promoting muscle repair or muscle regeneration, reducing inflammation or symptoms thereof, and preventing or reducing skin damage or symptoms thereof.

### BACKGROUND OF THE INVENTION

In general, aging is understood to be a phenomenon in which the number of cells decrease with age, and physical function, physiological function, and mental function decline. Aging comes with wrinkles in the skin, loss of hair and teeth, loss of vision and hearing, loss of motor function, loss of bone mass, etc. Aging is not defined as a disease, but a decrease in physical function and physiological function which increases the risk of so-called geriatric diseases.

Muscle degeneration or muscle atrophy is the decline of the skeletal muscle mass which can be partial or complete. It can be caused by a number of aspects which includes aging, lack of physical activity, malnutrition, and genetics. Muscle atrophy or degeneration is common in athletes. It occurs as a consequence of over-use, trauma, immobilization or lack of use after sports injuries where the strength of the muscle is lost. In general, muscles have adequate repair capacity particularly in young people, but this repair process can become ineffective after repeated rounds of over-use, severe trauma or other processes. In such cases the muscles lose function and strength of contraction and can be replaced by scar tissue. The scar tissue lacks contractility and causes loss of muscle function.

A metabolic disorder occurs when abnormal chemical reactions in your body disrupt the process your body uses to get or make energy from the food you eat. There are different groups of disorders. Some affect the breakdown of amino acids, carbohydrates, or lipids. Another group, mitochondrial diseases, affects the parts of the cells that produce the energy. Metabolic disorders include a group of pathologies such as insulin resistance, fatty liver, dyslipidemia, and hypertension leading to the development of type 2 diabetes and heart failure.

NAD+, and its reduced form NADH, are best known for their roles as coenzymes in redox reactions, linking the catabolic reactions of glycolysis and the TCA cycle to oxidative phosphorylation. In the last two decades, however, another role for NAD+ has been uncovered. Perhaps equally as important (and ancient) is NAD's role as a signaling molecule. From plants to metazoans, an increase in intracellular levels of NAD+ directs cells to make adjustments to ensure survival, including increasing energy production and utilization, boosting cellular repair, and coordinating circadian rhythms.

NAD+ levels are converted to signals by various enzymes that have evolved to sense NAD+, including the sirtuin deacylases (SIRT1-SIRT7), CtBPs, and poly-ADP-ribose polymerases (PARPs). They can sense NAD+ fluctuations because, unlike the enzymes of glycolysis and the TCA cycle, their dissociation constants for NAD+ are near physiological concentrations. Unfortunately, NAD+ levels steadily decline during aging. By the time a mouse or human is middle aged, levels of NAD+ have fallen to half of youthful levels, with resulting loss of sirtuin and PARP activity.

Conventional nutraceutical compositions are not necessarily sufficient in terms of maintaining a healthy body condition, aging and further improvements are necessary. In addition, current therapies are not specifically directed toward basic mechanisms of muscle cell repair and regeneration.

Based on the above, there is a need in developing new compositions and methods that augment the nutritional requirements of a subject, thereby reducing the symptoms of aging and maladies as noted above.

The above mentioned shortcomings, disadvantages and problems are addressed herein, which will be understood by reading and studying the following specification.

### SUMMARY OF THE INVENTION

Provided herein are compositions comprising: (i) an NAD+ precursor; (ii) at least one stilbenoid; (iii) hyaluronic acid; (iii) and collagen peptides. In embodiments, the compositions disclosed herein include a pharmaceutically acceptable carrier.

Also provided are methods that include administering to a subject in need the compositions disclosed herein for reducing aging and the symptoms thereof, increasing longevity, optimizing cellular repair, maintaining healthy mitochondria, improving muscle repair or regeneration, preventing or treating a metabolic disorder or symptoms thereof, preventing or reducing inflammation or symptoms thereof, and for preventing or reducing skin damage or symptoms thereof in a subject.

### DETAILED DESCRIPTION OF THE INVENTION

The present invention relates to a compositions for reducing aging and the symptoms thereof, increasing longevity, optimizing cellular repair, maintaining healthy mitochondria and method of repairing and regenerating muscles in human. In particular, the present invention relates to a composition comprising nicotinamide mononucleotide, at least one stilbenoid, collagen peptides and hyaluronic acid.

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%,1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The terms “nutraceutical”, “supplement” and “nutritional supplement” are used interchangeably herein to refer to a composition which, when administered to a subject (human) induces a desired effect. Included are derivatives and analogs of those compounds or classes of compounds specifically mentioned which also induce the desired effect.

The term “ageing” or “aging” when used herein refer to or describe the changes both structural and functional that accumulate in an organism as a result of molecular and cellular damage over time. Symptoms of aging include, without limitation, reduced physical function, low physiological function, mental function decline, wrinkles in the skin, loss of hair and teeth, loss of vision and hearing, loss of motor function, loss of bone mass.

The term “Nicotinamide mononucleotide” also termed as NMN, NAMN, β-NMN, beta-Nicotinamide mononucleotide, Nicotinamide ribotide, beta-NMN, Nicotinamide D-ribonucleotide, Nicotinamide ribonucleotide, Nicotinamide nucleotide.

The term “Stilbenoids” also termed as stilbenes or Proteasome Inhibitors.

The term “Resveratrol” also termed as RSV, trans-resveratrol, 3,4′, 5-Trihydroxystilbene, 3,5,4′-Trihydroxystilbene, 3,4′, 5-Stilbenetriol or 3,4′, 5-Trihydroxy-trans-stilbene.

The term “Pterostilbene” also termed as 3′, 5′-Dimethoxy-4-stilbenol, 3,5-Dimethoxy-4′-hydroxy-E-stilbene, 3′, 5′-Dimethoxy-resveratrol or trans-pterostilbene,

The term “Hyaluronic acid” also termed as hyaluronan.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

The term “infection” or “infectious disease” refers to a disease or condition that can be caused by organisms such as a bacterium, virus, fungi or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the disease is Bacterial vaginosis (i.e., bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e., a bacterial inflammation of the meninges); Bacterial pneumonia (i.e., a bacterial infection of the lungs); Urinary tract infection; Bacterial gastroenteritis; or Bacterial skin infections (e.g., impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins. Coronavirus disease 2019 (COVID-19) is an infectious disease caused by severe acute respiratory syndrome coronavirus 2 (SARS-CoV-2).

In various configurations, a nutraceutical composition of the present invention can be a formulation including a pill, a tablet, a caplet, a capsule, a chewable tablet, a quick dissolve tablet, a powder, an effervescent tablet, a non-aqueous liquid, an aqueous liquid, a granule, a suspension, a solution, an emulsion, a syrup, a jelly, a paste, or a powder.

“Carriers” or “vehicles” as used herein refer to carrier materials suitable for the administration of the composition. Carriers and vehicles useful herein include any such materials known in the art, e.g., any liquid, gel, solvent, liquid diluent, solubilizer, or the like, which is nontoxic and which does not interact with other components of the composition in a deleterious manner.

“Pharmaceutically acceptable” as used herein refers to a material that is non-toxic, not biologically or otherwise undesirable. The material can be administered to a subject, along with the composition disclosed herein, without causing any undesirable biological effects. The carrier is selected to minimize the degradation of the active ingredient. It even minimizes any adverse side effects in the subject.

The term “dosage form” is intended to include both solid and liquid formulations of the composition and one skilled in the art will appreciate that an active ingredient can exist in different preparations depending on the desired dose and pharmacokinetic parameters.

Solid forms include tablets, capsules, granules, and bulk powders. The dry forms of the supplements are typically taken in conjunction with water or diluted with water. When diluted with water this factor must be considered when preparing the mix. Tablets may contain suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules and effervescent preparations reconstituted from effervescent granules. Such liquid oral dosage forms may contain, for example, suitable solvents, preservatives, emulsifying agents, suspending agents, diluents, sweeteners, melting agents, and colouring and flavouring agents.

The term “subject” refers to a mammal. Mammals include, but are not limited to, humans, non-human primates, horses, dogs and cats.

### Compositions

The invention as noted above, involves methods and compositions related to treatment of aging and to commence muscle repair in a subject by administering to the subject (e.g., orally administering to the subject) a composition comprising a NAD+ precursor, one or more stilbenoids, collagen peptides and hyaluronic acid. This composition also helps in promoting cellular rejuvenation and mitochondrial function.

The compositions provided herein are capable of cellular rejuvenation, promoting muscle repair, well-being, healthy aging, longevity, optimizing cellular repair, maintaining healthy mitochondria and aiding preventative health management.

Nicotinamide mononucleotide or “NMN” or “NAMN” (chemical formula: C₁₁H₁₅N₂O₈P) is a bioactive nucleotide which is produced in the body of many organisms including human. NMN is generally identified as an intermediate metabolite involved in the biosynthesis of coenzyme NAD+. It is a compound that can rapidly increase cellular NAD+ concentration. In embodiments, NMN is at least 90% pure. In embodiments, NMN is at least 95% pure. A structure of NMN is provided below.

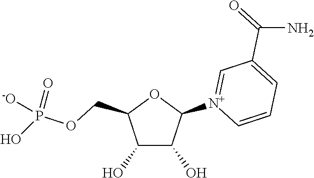

There are two optical isomers of nicotinamide mononucleotide, an α-form and a β-form. In embodiments, for nutritional food, supplements, and beverages NMN is used in the β-form. Nicotinamide mononucleotide may also be synthetically prepared. For example, NMN may be prepared by reacting nicotinamide or its analogues or derivatives with L-ribose tetraacetate. In addition, nicotinamide mononucleotides are commercially available.

Provided herein are compositions for administering to a subject an effective amount of nicotinamide mononucleotide. NMN can be administered in single dosage units that include about 30 mg, about 40 mg, about 50 mg, about 60 mg, about 70 mg, about 80 mg, about 90 mg, about 100 mg, about 150 mg, about 200 mg, about 300 mg, about 400 mg, and about 500 mg. In embodiments, a single dosage unit comprises about 30mg to about 500 mg NMN. In embodiments, a single dosage unit comprises about 100 to about 300 mg NMN. In embodiments, a single dosage unit contains about 200 milligrams of Nicotinamide mononucleotide.

Stilbenoids or stilbenes or proteasome inhibitors are naturally occurring phytochemicals. Stilbenoids bear the core structure of 1,2-diphenylethylene. These compounds are stress metabolites, produced in leaves and sapwood of plants in response to fungal infection. Though known as plant defense compounds, these phytochemicals have an enormous diversity of effects on biological and cellular processes applicable to human health.

Resveratrol or 3,5,4′-trihydroxy-trans-stilbene (chemical formula C₁₄H₁₂O₃) is one of the most common stilbenoids which is found in the red grapes skin, peanuts and berries. Resveratrol has been shown to extend the life of yeast, presumably due to the activation of SIR2 gene, which is the longevity gene family (sirtuin-family). A structure of resveratrol is provided below.

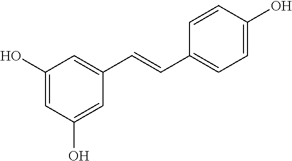 [SMILES: Oc1ccc(/C=C/c2cc(O)cc(O)c2)cc1]

Resveratrol can include derivatives such as methylated, methoxylated, hydroxylated or halogenated resveratrol, trans isomers, cis isomers and dimers. In embodiments, heat-stable trans isomers are used for supplements, health food and beverages. Resveratrol may also be synthetically prepared, for example by extracting and purifying a starting material wherein, starting material can be 3,5-dihydroxybenzoic acid. It can also be prepared by Heck-Mizoroki CC cross-coupling reaction. Also, resveratrol is commercially available.

Pterostilbene or trans-3,5-dimethoxy-4-hydroxystilbene (Chemical formula C₁₆H₁₆O₃) is also a stilbenoid which is structurally similar to resveratrol. It is a polyphenol which occurs in plants, particularly blue berries and almonds. It serves as a powerful antioxidant. It has an increased bioavailability in comparison to other stilbenoids due to the presence of two methoxy groups which cause it to exhibit increased lipophilic and oral absorption. It reduces oxidative stress and the production of reactive oxygen species, such as hydrogen peroxide and superoxide. A structure of pterostilbene is provided below.

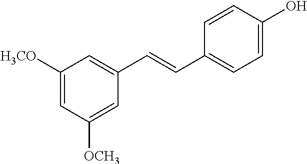 [SMILES: COc1cc(/C=C/c2ccc(O)cc2)cc(OC)c1]

In embodiments, compositions disclosed herein can include pterostilbene and/or resveratrol and single dosage units of the compositions can include about 10 mg, about 20 mg, about 30 mg, about 40 mg, about 50 mg, about 60 mg, about 70 mg, about 80 mg, about 100 mg, about 200 mg, about 300 mg, about 400 mg, and about 500 mg of pterostilbene and/or resveratrol. In embodiments, a single dosage unit includes about 10 mg to about 500 mg pterostilbene and/or resveratrol. In embodiments, a single dosage unit includes about 50 mg to about 200 mg pterostilbene and/or resveratrol. In embodiments, a single dosage unit includes about 100 milligrams of Pterostilbene and/or 100 milligrams of Resveratrol.

Pterostilbene and Resveratrol are anti-oxidants and regulate oxidative damage, inflammation, telomere attrition and cell senescence in a human body. The anti-oxidative effect of these compounds depend on redox properties of its phenolic hydroxyl group and the potential for electron delocalization across the conjugated structure. Pterostilbene and resveratrol play a major role in increasing SIRT1 activity. Sirtuins are a class of proteins which are involved in regulating cellular health. They regulate intracellular pathways via the activation of transcription factors. They help in activation of enzymes which are responsive to nutrient availability. SIRT1 is a class III protein deacetylase that regulates metabolic activity in response to cellular stress. It promotes cell survival by inhibiting apoptosis.

A combination of pterostilbene and resveratrol can increase the overall effectiveness of the composition. Resveratrol with pterostilbene can promote new blood vessel growth, healthy mitochondria energy function and efficient ATP production. Both compounds have cytoprotective effect and activate AMPK (5′ adenosine monophosphate-activated protein kinase) which is an enzyme that plays a role in cellular energy production, regulating growth and reprogramming metabolism. The compounds also stimulate SIRT1 which is the enzyme that deacetylates proteins that contribute to cellular regulation. The combination of pterostilbene and resveratrol promote healthy aging, weight loss, and skin health. Reduced oxidative stress and a properly modulated inflammatory response appear to be a major key in proper health. Antioxidants and natural anti-inflammatory agents in pterostilbene and resveratrol are powerful natural ways to promote these healthy aging properties.

In order to maintain a healthy lifestyle it is recommended to exercise. However, exercise also leads to oxidative stress, inflammation and muscle injury. In the case of athletes the physical activity level is much higher in comparison to ordinary human being. It becomes necessary to modulate the physical performance and prevent oxidative stress in the case of athletes. Resveratrol and pterostilbene delay fatigue by hindering lipid peroxidation and have protective effect on liver glycogen as the glycogen reserves deplete during physical activity. This can help athletes to regain muscle strength and improve physical performance.

Collagen is an essential component of skin and is responsible for the elasticity of the skin. The common feature for all collagens is a sequence that can be expressed as (Gly-X-Y)*n, where X and Y are frequently represented by proline (Pro) and hydroxyproline (Hyp), respectively. This sequence is necessary for the collagen to assemble the fibrils that subsequently form fibers, providing structural integrity for the extracellular matrix of conjunctive tissues. Collagen can be hydrolysed to yield peptides, which can be more readily ingested in various forms. These forms can be as a supplement, functional food, or beverage. The peptides are characterized by excellent cold-water solubility and, even in highly concentrated solutions, they do not form a gel. The compositions provided herein can include collagen peptides. In embodiments, a single dosage unit includes about 5mg, about 10 mg, about 20 mg, about 50 mg, about 100 mg, about 200 mg, about 300 mg, about 400 mg, or about 500 mg collagen peptides. In embodiments, a single dosage unit can include about 5 mg to about 500 mg of collagen peptides. In embodiments, a single dosage unit can include about 10 mg to about 200 mg of collagen peptides. In embodiments, a single dosage unit can include about 25 mg to about 100 mg of collagen peptides. In embodiments, a single dosage unit includes about 50 milligrams of collagen peptides.

Hyaluronic acid (HA) or hyaluronan is a naturally occurring, water soluble polysaccharide, specifically a glycosaminoglycan, which is a component of the extra-cellular matrix and is widely distributed in animal tissues. The largest amounts of hyaluronic acid are found in the skin, connective tissues and eyes. Hyaluronic acid has crucial functions in restoration of epithelium. Hyaluronic acid binds water and therefore helps in retaining skin moisture. It helps to combat aging by providing its adequate amount to moisture as dry and flaky skin tend to age early. In embodiments, a single dosage unit includes about 0.25 mg, about 0.5 mg, about 1 mg, about 2 mg, about, 5 mg, about 10 mg, about 20 mg, about 50 mg, about 100 mg, about 200 mg, about 300 mg, about 400 mg, or about 500 mg HA. In embodiments, a single dosage unit can include about 0.25 mg to about 500 mg HA. In embodiments, a single dosage unit can include about 1 mg to about 200 mg of HA. In embodiments, a single dosage unit can include about 5 mg to about 100 mg of collagen peptides. In embodiments, a single dosage unit includes about 10 mg to about 50 mg HA. In embodiments, a single dosage unit of the composition includes about 25 milligrams of HA. Orally administered hyaluronic acid can be absorbed in the digestive tract and migrates to the relevant connective tissues. Alternatively, orally administered hyaluronic acid can have a biological effect without being absorbed. HA has excellent biocompatibility and does not cause allergic reactions when applied to a subject. In addition, HA has the ability to bind to large amounts of water, making it an excellent volumizer of soft tissues.

The active ingredients of the composition of this invention have different mechanisms of action thus providing synergistic effects in the treatment or prevention of the diseases or conditions specified above.

In some embodiments, the compositions can include at least one excipient. In embodiments, the at least one excipient is a bulking agent, a dissolution agent, a wetting agent, a lubricant, a colouring, a flavouring, a disintegrant, a coating, a binder, an antioxidant, a taste masking agent, or a sweetener.

Examples of excipients includes lactose, sucrose, fructose, glucose, glucose hydrate, sucrose, refined sucrose, xylitol, sorbitol, mannitol, palatinose, reduced palatinose, powdered reduced maltose, water candy, carmellose, dextrin, corn starch , gelatinized starch, potato starch, corn starch, hydroxypropyl starch, amino acid, kaolin, silica, silica, aluminum silicate, sodium bicarbonate, calcium phosphate, calcium dihydrogen phosphate, calcium carbonate, oxidized magnesium, aluminum hydroxide, fatty acids or salts thereof, fatty acid glycerides, olive oil, soybean oil, corn oil, fatty oils, propylene glycol, ethylene glycol, polyethylene glycol, glycerol and the like.

Examples of binders include, but are not limited to, crystalline cellulose, crystalline cellulose/carmellose sodium, methylcellulose, hydroxypropyl cellulose, low substituted hydroxypropyl cellulose, hydroxypropyl methylcellulose, hydroxypropyl methylcellulose phthalate, hydroxypropyl methylcellulose acetate succinate, carmellose sodium, ethyl cellulose carboxymethylethyl cellulose, hydroxyethyl cellulose, wheat starch, rice starch, corn starch, potato starch, pregelatinized starch, partially pregelatinized starch, hydroxypropyl starch, dextrin, pullulan, polyvinyl pyrrolidone, aminoalkyl methacrylate copolymer E, aminoalkyl methacrylatecopolymers RS, methacrylic acid copolymer L, methacrylic acid copolymer, polyvinyl acetal diethylamino acetate, polyvinyl alcohol, gum arabic, gum arabic powder, agar, gelatin, white shellac, tragacanth and macrogol.

Examples of lubricants include, but are not limited to, magnesium stearate, calcium stearate, stearic acid, hydrous silicon dioxide, sucrose fatty acid ester, wheat starch, rice starch, corn starch, synthetic aluminum silicate, dried aluminum hydroxide gel, metasilicic acid, magnesium aluminate, calcium hydrogen phosphate, anhydrous calcium hydrogen phosphate, hydrogenated vegetable oil, polyethylene glycol, light anhydrous silicic acid, synthetic aluminum silicate, macrogol, talc and the like.

Examples of disintegrants include, but are not limited to, crystalline cellulose, methyl cellulose, low substituted hydroxypropyl cellulose, carmellose, wheat starch, rice starch, corn starch, potato starch, gelatinized starch, hydroxypropyl starch, sodium carboxymethyl starch, tragacanth and the like.

Examples of the emulsifiers include, but are not limited to, soybean lecithin, sucrose fatty acid ester, polyoxyl stearate, hydrogenated castor oil polyoxyethylene, polyoxyethylene polyoxypropylene glycol, sorbitan sesquioleate, sorbitan trioleate, sorbitan monostearate, monopalmitic acid sorbitan, sorbitan monolaurate, polysorbate, glycerin monostearate, sodium lauryl sulfate, lauromacrogol and the like.

Examples of solubilizers include, but are not limited to, sodium phosphate, polysorbates, and as the pH adjuster, citric acid, sodium citrate, acetic acid, tartaric acid, sodium hydroxide, potassium hydroxide, sodium hydrogen carbonate, sodium carbonate and lactic acid.

Examples of the antioxidants include, but are not limited to, ascorbic acid, erythorbic acid and catechin.

In embodiments, the composition is provided in a beverage, such as a sports drink, or a concentrate thereof. Other components can include, but are not limited to, cranberry juice, orange juice, acerola juice, grape juice, apple juice, peach juice, peach juice, pineapple juice, litchi juice, raspberry juice, pomegranate juice, kiwi juice, cherry juice, blueberry juice. In addition, acai puree, mango puree, apricot paste, alonia extract, fermented plant extract, grape peel extract powder, grape seed extract powder, grape sprout extract powder, pomegranate seed extract powder, French coast pine bark extract powder, ginger powder extract, etc.

The compositions disclosed herein can be used therapeutically in combination with a pharmaceutically acceptable carrier.

Pharmaceutically acceptable carriers for use in the disclosed invention can be selected based upon a number of factors. The factors may include the active compound used, and its concentration, stability and intended bioavailability of the compounds. It even depends on the subject, its age, height, weight, health conditions and the route of administration.

Preferably, the preparations are in unit dosage form. In such form, the preparation is subdivided into unit doses containing appropriate quantities of the active components. The unit dosage form can be a packaged preparation, the package containing discrete quantities of preparation, for example, packeted tablets, capsules, and powders in vials or ampoules. The unit dosage form can be in beverage form or beverage concentrate. The unit dosage form can also be a capsule, cachet, or tablet itself or it can be the appropriate number of any of these in packaged form.

The quantity of active compound in a unit dose of preparation may be varied according to the particular application and the potency of the active ingredients.

### Methods

Provided herein are methods for improving the health of a subject either as a treatment or prophylactically. Provided herein are methods for improving muscle repair or regeneration in a subject in need thereof, by administering an effective amount of any of the compositions disclosed herein. In embodiments, the subject in need has a physical injury or accident, muscle immobilization, muscle overuse, loss of blood circulation, or lack of muscle use after injury.

Provided herein are methods for reducing symptoms of aging in a subject in need thereof, by administering an effective amount of any of the compositions disclosed herein.

Provided herein are methods for preventing or treating a metabolic disorder or symptoms thereof in a subject in need thereof, by administering an effective amount of any of the compositions disclosed herein. Metabolic disorders include metabolic syndrome characterized by a group of pathologies which include insulin resistance, fatty liver, dyslipidemia, and hypertension leading to the development of type 2 diabetes and heart failure.

Provided herein are methods for preventing or reducing inflammation or symptoms thereof in a subject in need thereof, by administering an effective amount of any of the compositions disclosed herein. In embodiments, the inflammation is related to a viral infection. In embodiments, the viral infection is SARS-CoV-2.

Provided herein are methods improving neural function in a subject in need thereof, by administering an effective amount of any of the compositions disclosed herein.

Provided herein are methods for preventing or reducing skin damage or symptoms thereof in a subject in need thereof, by administering an effective amount of any of the compositions disclosed herein.

### P EMBODIMENTS

P1. A composition comprising nicotinamide mononucleotide (NMN), at least one stilbenoid, collagen peptides, and hyaluronic acid.
P2. The composition of embodiment P1, wherein the stilbenoid is resveratrol or pterostilbene.
P3. The composition of embodiment P1, comprising resveratrol and pterostilbene.
P4. A single dosage unit comprising the composition embodiment of Pl, comprising about 30 to about 300 mg of NMN.
P5. The single dosage unit of embodiment P4, comprising about 10 to about 100 mg Resveratrol or Pterostilbene.
P6. The single dosage unit of embodiment P4, comprising about 5 to about 50 mg collagen peptides.
P7. The single dosage unit of embodiment P4, comprising about 2.5 to about 25 mg hyaluronic acid.
P8. The composition of any of embodiments P1 to P3 or the single dosage unit of any of claims P4 to P7, comprising a pharmaceutically acceptable carrier.
P9. The composition or single dosage unit of embodiment P8, wherein the pharmaceutically acceptable carrier comprises a tablet, capsule, jelly, liquid, paste, pill, granule, or powder.
P10. A method for improving muscle repair or regeneration in a subject in need thereof, said method comprising administering an effective amount of any of the compositions or single dosage units of embodiments P8 or P9.
P11. The method of embodiment P10, wherein said subject in need thereof has a physical injury or accident, muscle immobilization, muscle overuse, loss of blood circulation, or lack of muscle use after injury.
P12. A method for reducing symptoms of aging in a subject in need thereof, said method comprising administering an effective amount of any of the compositions or single dosage units of embodiments P8 or P9.
P13. A method for preventing or treating a metabolic disorder or symptoms thereof in a subject in need thereof, said method comprising administering an effective amount of any of the compositions or single dosage units of embodiments P8 or P9.
P14. A method for preventing or reducing inflammation or symptoms thereof in a subject in need thereof, said method comprising administering an effective amount of any of the compositions or single dosage units of embodiments P8 or P9.
P15. The method of embodiment P14, wherein the inflammation is related to a viral infection.
P16. The method of embodiment P15, wherein the viral infection is SARS-CoV-2.
P17. A method of improving neural function in a subject in need thereof, said method comprising administering an effective amount of any of the compositions or single dosage units of embodiments P8 or P9.
P18. A method for preventing or reducing skin damage or symptoms thereof in a subject in need thereof, said method comprising administering an effective amount of any of the compositions or single dosage units of embodiments P8 or P9.
P19. The method of any of embodiments P10 to P18, wherein the subject is a mammal.
P20. The method of embodiment P19, wherein the subject is a human

### EXAMPLES

One skilled in the art would understand that descriptions of making and using the particles described herein is for the sole purpose of illustration, and that the present disclosure is not limited by this illustration.

### Example 1

A single capsule formulation is prepared containing 200 milligrams of Nicotinamide Mononucleotide, 100 milligrams of Resveratrol, 100 milligrams Pterostilbene, 50 milligrams of Collagen peptides and 25 milligrams of hyaluronic acid. The capsule includes a gelatin shell surrounding a fill material composed of the active compounds. The capsule is administered to a subject on a daily basis for treating aging, providing cells and mitochondria with essential nutrients to boost NAD+production, optimizing cellular repair, maintaining healthy mitochondria, and promoting a healthier and longer lifespan.

### Example 2

Single dose beverage concentrates are prepared containing 200 milligrams of Nicotinamide Mononucleotide, 100 milligrams of Resveratrol, 100 milligrams Pterostilbene, 50 milligrams of Collagen peptides and 25 milligrams of hyaluronic acid. The beverage concentrate is diluted and the diluted beverage concentrate is consumed by the subject.

What is claimed is:

1. A composition comprising nicotinamide mononucleotide (NMN), at least one stilbenoid, collagen peptides, and hyaluronic acid.

2. The composition of claim 1, wherein the stilbenoid is resveratrol or pterostilbene.

3. The composition of claim 1, comprising resveratrol and pterostilbene.

4. A single dosage unit comprising the composition of claim 1, comprising about 30 to about 300 mg of NMN.

5. The single dosage unit of claim 4, comprising about 10 to about 100 mg Resveratrol or Pterostilbene.

6. The single dosage unit of claim 4, comprising about 5 to about 50 mg collagen peptides. acid.

7. The single dosage unit of claim 4, comprising about 2.5 to about 25 mg hyaluronic

8. The composition of claim 1, comprising a pharmaceutically acceptable carrier.

9. The composition of claim 8, wherein the pharmaceutically acceptable carrier comprises a tablet, capsule, jelly, liquid, paste, pill, granule, or powder.

10. A method for improving muscle repair or regeneration in a subject in need thereof, said method comprising administering an effective amount of the composition of claim 8.

11. The method of claim 10, wherein said subject in need thereof has a physical injury or accident, muscle immobilization, muscle overuse, loss of blood circulation, or lack of muscle use after injury.

12. A method for reducing symptoms of aging in a subject in need thereof, said method comprising administering an effective amount of the composition of claims 8.

13. A method for preventing or treating a metabolic disorder or symptoms thereof in a subject in need thereof, said method comprising administering an effective amount of the composition of claim 8.

14. A method for preventing or reducing inflammation or symptoms thereof in a subject in need thereof, said method comprising administering an effective amount of the composition of claim 8.

15. The method of claim 14, wherein the inflammation is related to a viral infection.

16. The method of claim 15, wherein the viral infection is SARS-CoV-2.

17. A method of improving neural function in a subject in need thereof, said method comprising administering an effective amount of the composition of claim 8.

18. A method for preventing or reducing skin damage or symptoms thereof in a subject in need thereof, said method comprising administering an effective amount of the composition of claim 8.

19. The method of any of claim 10, wherein the subject is a mammal.

20. The method of claim 19, wherein the subject is a human.

21. A method for improving muscle repair or regeneration in a subject in need thereof, said method comprising administering an effective amount of the single dosage unit of claim 9.

22. The method of claim 21, wherein said subject in need thereof has a physical injury or accident, muscle immobilization, muscle overuse, loss of blood circulation, or lack of muscle use after injury.

23. A method for reducing symptoms of aging in a subject in need thereof, said method comprising administering an effective amount of the single dosage unit of claim 9.

24. A method for preventing or treating a metabolic disorder or symptoms thereof in a subject in need thereof, said method comprising administering an effective amount of the single dosage unit of claim 9.

25. A method for preventing or reducing inflammation or symptoms thereof in a subject in need thereof, said method comprising administering an effective amount of the single dosage unit of claim 9.

26. The method of claim 25, wherein the inflammation is related to a viral infection.

27. The method of claim 26, wherein the viral infection is SARS-CoV-2.

28. A method of improving neural function in a subject in need thereof, said method comprising administering an effective amount of the single dosage unit of claim 9.

29. A method for preventing or reducing skin damage or symptoms thereof in a subject in need thereof, said method comprising administering an effective amount of the single dosage unit of claim 9.

30. The method of claim 29, wherein the subject is a mammal.

31. The method of claim 30, wherein the subject is a human.
